(19) United States
(12) Patent Application Publication
(10) Pub. No.: US 2024/0315716 A1
(43) Pub. Date: Sep. 26, 2024

(54) TRANSVASCULAR ASPIRATION CATHETERS AND METHODS OF USE

(71) Applicant: Covidien LP, Mansfield, MA (US)

(72) Inventors: Raymond G. Ryan, Barna (IE); John Myles Curley, Galway (IE); Julie Bu, Galway (IE); Lloyd Johnston, Galway City (IE); Enda Boland, Moate (IE)

(21) Appl. No.: 18/608,651

(22) Filed: Mar. 18, 2024

Related U.S. Application Data
(60) Provisional application No. 63/491,260, filed on Mar. 20, 2023.

(51) Int. Cl. A61B 17/221; A61B 17/22; A61M 25/00

(52) U.S. Cl. CPC A61B 17/221; A61B 2017/22079; A61M 25/0023

(57) ABSTRACT

Intravascular catheters are disclosed herein. According to some embodiments, the present technology includes a catheter comprising an elongate tubular sidewall defining a lumen extending therethrough and having a proximal end, a distal end, and a length between the proximal and distal ends. The sidewall can comprise a plurality of filaments, at least some of the filaments being interwoven with other filaments of the plurality of filaments. The catheter can be configured to be positioned around a turn in a blood vessel having a radius of curvature no greater than 24 mm, and wherein, while the distal end of the catheter is distal of the turn, the lumen remains at least 70 percent patent while torque applied to the sidewall at the proximal end of the catheter is transmitted to the distal end.

### CROSS-REFERENCE TO RELATED APPLICATIONS

This application claims the benefit of priority to U.S. Provisional Application No. 63/491,260, filed Mar. 20, 2023, which is incorporated herein by reference in its entirety.

### TECHNICAL FIELD

The present technology relates to aspiration catheters and methods of use. In particular, the present technology relates to aspiration catheters for use in the removal of occlusions.

### BACKGROUND

Many interventional procedures, such as mechanical thrombectomy, include removal of all or a portion of the targeted occlusion in a blood vessel via aspiration. Aspiration occurs through an elongate catheter shaft that is advanced through a patient's vasculature to a desired treatment location. For optimum performance, the catheter shaft must strike a balance between various performance metrics, including pushability (i.e., column strength), torquability (e.g., ability to translate torque from the proximal hub to the distal tip), kink resistance, and suction force. Existing aspiration catheter constructions fail to strike such a balance and are often ineffective at accessing and/or aspirating occlusive material. Accordingly, there exists a need for an aspiration catheter that can access a treatment location in a blood vessel and effectively aspirate occlusive material.

### SUMMARY

The subject technology is illustrated, for example, according to various aspects described below, including with reference to FIGS. 1A-6. Various examples of aspects of the subject technology are described as numbered examples (1, 2, 3, etc.) for convenience. These are provided as examples and do not limit the subject technology.
1. An intravascular catheter, comprising:
an elongate tubular sidewall defining a lumen extending therethrough and having a proximal end, a distal end, and a length between the proximal and distal ends, the sidewall comprising a plurality of filaments, at least some of the filaments being interwoven with other filaments of the plurality of filaments, wherein the catheter is configured to be positioned around a turn in a blood vessel having a radius of curvature no greater than 24 mm, and wherein, while the distal end of the catheter is distal of the turn, the lumen remains at least 70 percent patent while torque applied to the sidewall at the proximal end of the catheter is transmitted to the distal end.
2. The catheter of Example 1, wherein the torque is at least 12 Nm.
3. The catheter of Example 1 or Example 2, wherein the sidewall comprises a longitudinal axis extending along its length, and wherein torque applied at the proximal end of the catheter is configured to rotate the sidewall 360 degrees about the longitudinal axis.
4. The catheter of any one of Examples 1 to 3, wherein, when the catheter is positioned around the turn and the torque is applied while the lumen remains at least 70 percent patent, an inserted length of the catheter is at least 50 percent.
5. The catheter of any one of Examples 1 to 4, wherein the sidewall has an outer diameter of at least 8 Fr.
6. The catheter of any one of Examples 1 to 5, wherein the sidewall has an outer diameter of at least 12 Fr.
7. The catheter of any one of Examples 1 to 6, wherein the turn in the blood vessel has a bend angle that is greater than or equal to 120 degrees.
8. The catheter of any one of Examples 1 to 7, wherein the sidewall defines a lumen extending along the longitudinal axis, and wherein the proximal end of the catheter is configured to be fluidly coupled to a negative pressure source to aspirate occlusive material within the blood vessel into and through the lumen.
9. An intravascular catheter, comprising:
a tubular sidewall having a longitudinal axis, the sidewall comprising a first number of first wires and a second number of second wires, the second number greater than the first number, wherein:
   the first wires have a first cross-sectional area and are helically wrapped around the longitudinal axis in a first direction without crossing over one another, and
   the second wires have a second cross-sectional area less than the first cross-sectional area, wherein the second wires comprise (a) a first group wound in the first direction and that do not cross over the first wires, and (b) a second group wound in a second direction opposite the first direction, and wherein the wires in the second group are interwoven with the first wires and the first group of the second wires.
10. The catheter of Example 9, wherein the second number is at least five times greater than the first number.
11. The catheter of Example 9 or Example 10, wherein a cross-sectional shape of the first wires is different than a cross-sectional shape of the second wires.
12. The catheter of any one of Examples 9 to 11, wherein the first wires have a rectangular cross-sectional shape and the second wires have a circular cross-sectional shape.
13. The catheter of any one of Examples 9 to 12, wherein the sidewall defines a lumen, and wherein a proximal end of the catheter is configured to be fluidly coupled to a negative pressure source to aspirate occlusive material within the blood vessel into and through the lumen.
14. An intravascular catheter, comprising:
a proximal end, a distal end, and a longitudinal axis extending therebetween;
a tubular sidewall defining a lumen, the sidewall comprising a plurality of wires embedded in a material, the plurality of wires comprising a first number of first wires and a second number of second wires, the second number greater than the first number, wherein:
   the first wires are helically wrapped around the longitudinal axis in a first direction without crossing over one another, the first wires being configured to resist radial collapse of the sidewall, and
   the second wires comprise (a) a first group wound in the first direction and that do not cross over the first wires, and (b) a second group wound in a second direction opposite the first direction, wherein the second wires are configured to engage the first wires to resist radial expansion of the first wires, thereby providing improved torqability of the sidewall,
wherein the proximal end of the catheter is configured to be fluidly coupled to a negative pressure source for application of suction through the lumen.
15. The catheter of Example 14, wherein the material is a first material and the plurality of wires and first material together comprise a first layer, and wherein the sidewall further comprises a second layer radially inward of the first layer.
16. The catheter of Example 14, wherein the material is a first material and the plurality of wires and first material together comprise a first layer, and wherein the sidewall further comprises a second layer radially outward of the first layer.
17. The catheter of Example 14, wherein the material is a first material and the plurality of wires and first material together comprise a first layer, and wherein the sidewall further comprises a second layer radially inward of the first layer and a third layer radially outward of the first layer.
18. The catheter of any one of Examples 14 to 17, wherein a proximal end of the catheter is configured to be fluidly coupled to a negative pressure source to aspirate occlusive material within the blood vessel into and through the lumen.
19. The catheter of any one of Examples 14 to 18, wherein the tubular sidewall has an outer diameter of at least 8 Fr.
20 The catheter of any one of Examples 14 to 18, wherein the tubular sidewall has an outer diameter of at least 12 Fr.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a side view of a catheter configured in accordance with several embodiments of the present technology.

FIG. 1B is an axial cross-sectional view of the catheter in FIG. 1A, taken along line 1B-1B.

FIG. 2 is an axial cross-sectional view of a catheter configured in accordance with several embodiments of the present technology.

FIG. 3 is a side view of a portion of a catheter configured in accordance with several embodiments of the present technology.

FIGS. 4, 5 and 6 show different example use cases for the catheters of the present technology.

### DETAILED DESCRIPTION

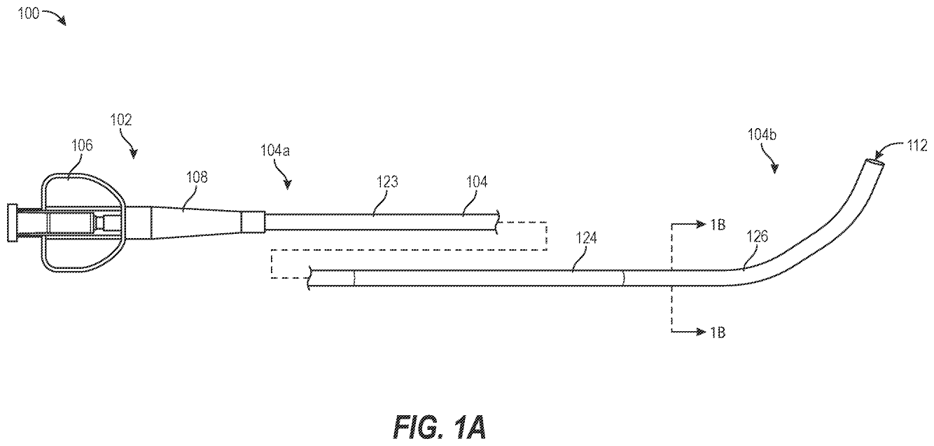

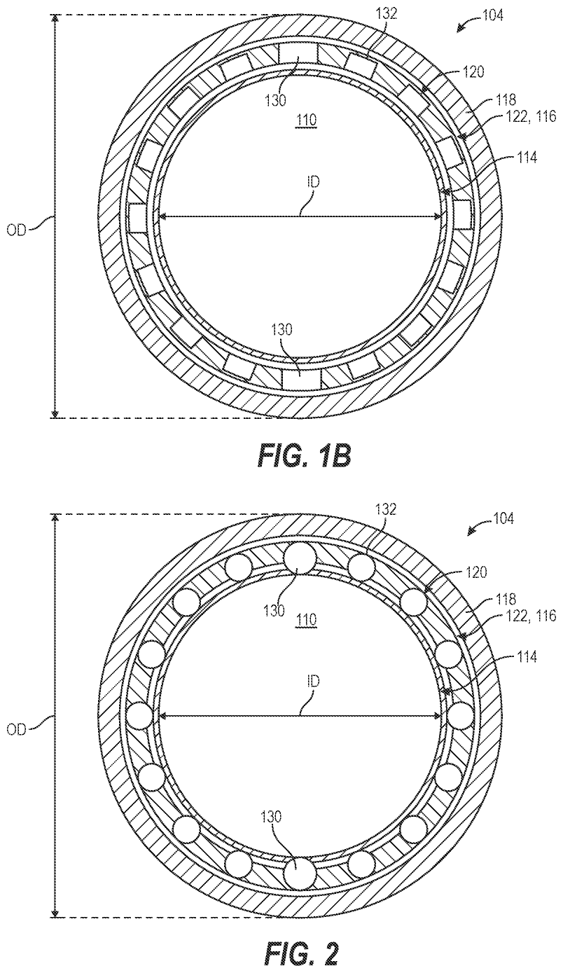

The present technology is directed to catheters and associated methods of use. Specific details of several embodiments of catheter devices, systems, and methods in accordance with the present technology are described below with reference to FIGS. 1A-6. Many embodiments of the present technology are particularly useful in treating targets located in tortuous and/or narrow vessels, such as certain sites in the neurovascular system, the pulmonary system, the peripheral vascular system, or the coronary vascular system. While the catheter constructions disclosed herein are described in the context of mechanical thrombectomy, the present technology can be used in other medical procedures. Likewise, while the catheter constructions of the present technology are described in the context of aspiration catheters, the present technology can be utilized in other types of catheters, including those not intended and/or suitable for aspiration, such as a guide catheter, support catheter, etc.

With regard to the terms “distal” and “proximal” within this description, unless otherwise specified, the terms can reference a relative position of the portions of a catheter and/or an associated device with reference to an operator and/or a location in the vasculature. Also, the term “thickness” as used herein with respect to a particular material or layer refers to the perpendicular distance between the plane running through and generally parallel with the radially outermost surface of the particular material or layer and the plane running through and generally parallel with the radially innermost surface of the particular material or layer.

FIG. 1A is a side view of a catheter 100 configured in accordance with several embodiments of the present technology, and FIG. 1B is a cross-sectional axial view taken along line 1B-1B in FIG. 1A. Referring to FIGS. 1A and 1B together, the catheter 100 includes a handle assembly 102 and an elongate shaft 104 having a proximal portion 104a coupled to the handle assembly 102 and a distal portion 104b. The handle assembly 102 includes a hub 106 configured to facilitate connection to a negative pressure source (not shown) and/other devices (e.g., a syringe, a Y-adapter, etc.) and a transition portion 108 configured to provide strain relief at the proximal portion 104a. In other embodiments, the handle assembly 102 can have other suitable configurations based on the desired functions and characteristics of the catheter 100.

The shaft 104 comprises a generally tubular sidewall having an inner surface that defines a lumen 110 (FIG. 1B) extending from the proximal portion 104a of the shaft 104 to an opening 112 at the distal terminus of the distal portion 104b. The lumen 110 can be configured to slidably receive and facilitate the passage therethrough of one or more medical devices, such as guidewires, balloon catheters, implants, intrasaccular occlusion devices (e.g., coils, expandable cages, expandable meshes, etc.), infusion devices, stents and/or stent-grafts, intravascular occlusion devices, clot retrievers, implantable heart valves, and other suitable medical devices and/or associated delivery systems. Additionally or alternatively, the lumen 110 is configured to receive one or more fluids therethrough, such as radiopaque dye, saline, drugs, and the like.

The size of the lumen 110 (or inner diameter of the shaft 104) can vary, depending on the desired characteristics of the catheter 100. When used for aspiration, the greater the inner diameter of the shaft 104, the greater the suction force that can be applied at the distal end. In those embodiments where the catheter 100 is configured for use in the pulmonary vasculature (e.g., for treating pulmonary embolism), the shaft 104 can have an inner diameter of about 0.118 inches (9 French) to about 0.263 inches (20 French), about 0.131 inches (10 French) to about 0.158 inches (12 French), about 0.131 inches (10 French) or greater, about 0.158 inches (12 French) or greater, or about 0.158 inches (12 French). In those embodiments where the catheter 100 is configured for use in the peripheral vasculature (e.g., for tracking arteriovenous loop grafts), the shaft 104 can have an inner diameter of about 0.066 inches (5 French) to about 0.105 inches (8 French), about 0.079 inches (6 French) to about 0.105 inches (8 French), or about 0.105 inches (8 French). Although the shaft 104 shown in FIG. 1A has a generally round cross-sectional shape, it will be appreciated that the shaft 104 can include other cross-sectional shapes or combinations of shapes. For example, the cross-sectional shape of the shaft 104 can be oval, rectangular, square, triangular, polygonal, and/or any other suitable shape and/or combination of shapes.

The outer diameter of the shaft 104 can be the same or vary along its length. For example, in the embodiment shown in FIGS. 1A and 1B, the shaft 104 has an outer diameter that is generally constant along its length. In some embodiments, the outer diameter of the shaft 104 decreases in a proximal to distal direction (either stepwise or continuously). In either case, the outer diameter of the shaft 104 can be selected for the desired use of the catheter 100. For example, in those embodiments where the catheter 100 is configured for use in the pulmonary vasculature (e.g., for treating pulmonary embolism), the shaft 104 can have an outer diameter of about 0.131 inches (10 French) to about 0.315 inches (24 French), about 0.158 inches (12 French) to about 0.315 inches (24 French), about 0.158 inches (12 French) or greater, or about 0.158 inches (12 French). In those embodiments where the catheter 100 is configured for use in the peripheral vasculature (e.g., at a forearm loop graft dialysis via arteriovenous permanent access) the shaft 104 can have an outer diameter of about 0.079 inches (6 French) to about 0.118 inches (9 French), or about 0.105 (8 French). In those embodiments where the catheter 100 is configured for use within small anatomies of the patient, such as the neurovasculature (e.g., to treat ischemic stroke) or coronary vasculature, the shaft 104 can have an outer diameter of about 0.053 inches (4 French) to about 0.079 inches (6 French), of about 0.017 inches to about 0.079 inches (6 French), about 0.017 inches, about 0.021 inches, or about 0.024 inches.

As shown in FIG. 1A, in some embodiments the shaft 104 can have a pre-formed bend at the distal portion 104b (e.g., the shaft 104 can be shape set to have a desired bend angle), for example to facilitate navigation through and around various turns in the vasculature. The portion of the shaft 104 comprising the pre-formed bend can be sufficiently rigid and/or resilient such that the bend angle is substantially maintained while the shaft 104 is being advanced/withdrawn through or otherwise manipulated within the vasculature. In some embodiments, the portion of the shaft 104 comprising the pre-formed bend includes a composite material that reduces vessel interaction force (in comparison to existing commercial catheters). The bend angle can be tailored to the specific medical application, such as for navigating the unique curvatures of the pulmonary arteries, iliofemoral veins, below-the-knee arteries, and others as described herein.

In some embodiments, the shaft 104 does not have a pre-formed bend.

The shaft 104 can be formed of a first layer 114, a second layer 116, and a third layer 118. The first layer 114 can be the radially innermost layer (thus surrounding and defining the lumen 110) and surrounded by the second layer 116, and the second layer 116 can be surrounded by the third layer 118. As such, in some embodiments the third layer 118 comprises the radially outermost layer of the shaft 104. As shown schematically in FIG. 1B, the second layer 116 can comprise a braid 120 embedded in a material 122. In some embodiments, the second layer 116 comprises only the braid 120 (and not the material 122) which can be positioned between the first and third layers 114, 118. In some examples where the second layer 116 comprises only the braid 120, the material of the third layer 118 can be disposed directly on and around the filaments of the braid 120 such that the braid 120 is embedded within the material of the third layer 118. In some embodiments, the shaft 104 comprises more or fewer than three layers (e.g., two layers, four layers, five layers, etc.).

The first layer 114 can extend from the proximal portion 104a of the shaft 104 to a location along the distal portion 104b of the shaft 104. For example, in the embodiment shown in FIGS. 1A and 1B, the first layer 114 extends from the proximal portion 104a of the shaft 104 to the opening 112 at the distal terminus of the distal portion 104b (e.g., the entire length of the shaft 104 or substantially the entire length of the shaft 104). In other embodiments, the first layer 114 extends along only a portion of the length of the shaft 104 and/or has a proximal and/or a distal terminus that does not correspond to a proximal terminus and/or a distal terminus, respectively, of the shaft 104. The length of the first layer 114 can vary depending upon, for example, the length of the shaft 104 and the desired characteristics and functions of the catheter 100.

The first layer 114 can be made of any suitable polymer (and/or combination of multiples polymers) and by any suitable process. In some embodiments, the first layer 114 comprises a lubricious polymer such as HDPE or polytetrafluoroethylene (PTFE), for example, or platinum, polyether-ether ketone (PEEK), polyethylene (PE), polypropylene (PP), or a copolymer of tetrafluoroethylene, such as FEP, a copolymer of tetrafluoroethylene with perfluoroethers, such as perfluoroalkoxy alkanes (PFA) (more specifically, perfluoropropyl vinyl ether or perfluoromethyl vinyl ether), or the like. Additional suitable polymers include, for example, polyoxymethylene (POM), polybutylene terephthalate (PBT), polyether block ester, polyether block amide (PEBA), fluorinated ethylene propylene (FEP), polyvinylchloride (PVC), polyurethane, polyimide, polyamide, polyphenylene sulfide (PPS), polyphenylene oxide (PPO), polysulfone, nylon, perfluoro(propyl vinyl ether) (PFA), polyether-ester, platinum, polymer/metal composites, Pebax® 2533, Pebax® 3533, Pebax® 4533, Pebax® 5533, Pebax® 6333 or Pebax® 7233, etc., or mixtures, blends or combinations thereof, and may also include or be made up of a lubricious polymer having a low coefficient of friction. In some embodiments (not shown), the first layer 114 includes one or more metals or metal alloys and/or combinations thereof. In a particular embodiment, the first layer 114 does not include any polymer material and solely comprises a metal and/or metal alloy.

As best shown in FIG. 1B, the third layer 118 directly contacts at least an outer surface of the second layer 116. The third layer 118 extends distally from the proximal portion 104a of the shaft 104 to a location along the distal portion 104b of the shaft 104 (e.g., the entire length of the shaft 104 or substantially the entire length of the shaft 104). The length of the third layer 118 can vary depending on, for example, the length of the shaft 104 and the desired characteristics and functions of the catheter 100. In some embodiments, the third layer 118 extends substantially the entire length of the shaft 104. In other embodiments, the third layer 118 extends along only a portion of the length of the shaft 104 and/or has a proximal and/or distal terminus that does not correspond to a proximal terminus and/or distal terminus, respectively, of the shaft 104.

The third layer 118 (and/or portions thereof) can be made of any suitable polymer (or composites or combinations thereof) and by any suitable process. Suitable polymers can include, for example, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysulfone, nylon, perfluoro(propyl vinyl ether) (PFA), polyether-ester, Pebax® 2533, Pebax® 3533, Pebax® 4533, Pebax® 5533, Pebax® 6333 or Pebax® 7233, platinum, polymer/metal composites, etc., or mixtures, blends or combinations thereof. In several embodiments, the third layer 118 is or at least includes a lubricious polymer and/or a hydrophilic coating to facilitate advancement of the shaft 104 through a larger catheter and/or the vasculature. In some embodiments (not shown), the third layer 118 includes one or more metals or metal alloys (combinations thereof). In a particular embodiment, the third layer 118 does not include any polymer material and solely comprises a metal and/or metal alloy.

In some embodiments, the stiffness of the third layer 118 (and/or the shaft 104) varies along its length. In such embodiments, the stiffness variation may be continuous or stepped by varying the size, shape, thickness, and/or material composition of the third layer 118. For example, in the embodiment shown in FIGS. 1A and 1B, the third layer 118 includes at least three unique portions along its length (labeled proximal to distal as first, second, and third portions 123, 124, and 126 respectively) in which the respective stiffnesses of the portions 123, 124, 126 decrease sequentially in a proximal to distal direction. For example, the first portion 123 has a first stiffness and the second portion 124 has a second stiffness less than the first stiffness, and the third portion 126 has a third stiffness less than the second stiffness. In other embodiments, the stiffness of the third layer 118 and/or the stiffnesses of the individual portions 123, 124, 126 can increase in a proximal to distal direction (e.g., the second portion 126 can be stiffer than the first portion 124, etc.), or be generally uniform in a proximal to distal direction. In other embodiments, the third layer 118 can have more or fewer portions having different stiffnesses (e.g., one continuous portion, three portions, four portions, five portions, etc.).

The first, second, and third portions 123, 124, and 126 can comprise the same or different materials. In some variations, one or more portions of the third layer 118 can be more transparent than one, some, or all of the other portions of the third layer 118 for better visualization of the portion. For example, in some embodiments the second portion 124 of the third layer 118 can be substantially transparent or translucent while the first and third portions 123, 126 are substantially opaque. In certain embodiments, the third portion 126 is more transparent than the first and second portions 123, 124. In some variations, the first, second, and third portions 123, 124, 126 have the same degree of transparency.

It will be appreciated that while the portions 123, 124, 126 of the third layer 118 are described herein as separate components with respect to the illustrated embodiments, the portions 123, 124, 126 can be provided as a single layer or structure. For example, the first and second portions 123, 124, 126 may be provided separately, but attached or combined together to physically form a single layer (e.g., a single homogeneous material).

Referring still to FIGS. 1A and 1B, the third layer 118 may be on and around the second layer 116, and the second layer 116 may be on and around the first layer 114. In some embodiments, some or all of the filaments of the braid 120 of the second layer 116 directly contact at least a portion of the first layer 114, the third layer 118, or both. The second layer 116 can extend distally from the proximal portion 104a of the shaft 104 to a distal terminus aligned with or just proximal of the distal terminus of the shaft 104. In other embodiments, the second layer 116 extends the entire length of the shaft 104. The length of the second layer 116 can vary depending upon, for example, the length of the shaft 104 and the desired characteristics and functions of the catheter 100. The material 122 of the second layer 116 can be made of any suitable polymer (or composites or combinations thereof) and by any suitable process. Suitable polymers can include, for example, any of the polymers disclosed herein, including but not limited to Pebax® 2533, Pebax® 3533, Pebax® 4533, Pebax® 5533, Pebax® 6333 or Pebax® 7233. In some embodiments, the material 122 is flowed over the filaments of the braid 120 such that the material 122 flows over and between the filaments.

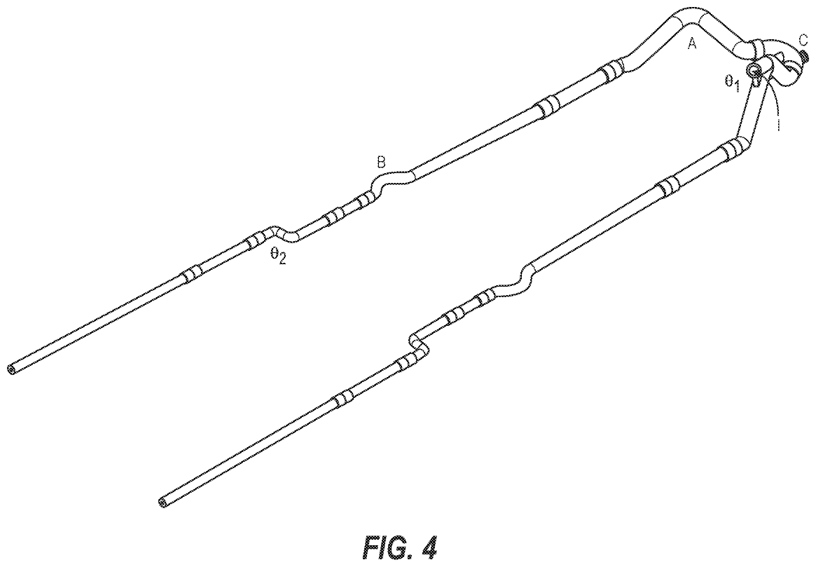

The braid 120 can be formed of a plurality of interwoven filaments (for example, as shown in FIGS. 1B, 2, and 3). The filaments of the braid 120 can comprise first filaments 130 and second filaments 132 (only a few labeled) interwoven with the first filaments 130. The first filaments 130 can advantageously be configured to resist radial collapse of the sidewall of the shaft 104 (thus providing improved resistance to kinking) and the second filaments 132 can be configured to engage the first filaments 130 to resist radial expansion of the first filaments 130 when torque is applied to the proximal end portion 104a of the shaft 104 (thus providing improved torque transmission along the sidewall with 1:1 torque control). Referring to FIGS. 1B and 3, the first filaments 130 can be helically wrapped around the longitudinal axis of the sidewall in a clockwise or counterclockwise direction without crossing over one another (e.g., thereby forming a coil). In some embodiments, for example as shown in FIG. 1B, the braid 120 can include two first filaments 130 spaced 180 degrees apart around a circumference of the shaft 104. In other embodiments, the braid 120 may include more than two first filaments 130 (e.g., three first filaments 130, four first filaments 130, etc.), so long as the size of the first filaments 130 and/or second filaments 132 is decreased to account for less space between adjacent turns of the first filaments 130 and such that none of the filaments 130, 132 longitudinally overlap. Interweaving the first and second filaments 130, 132 can beneficially decrease the overall thickness and outer diameter of the shaft 104 as compared to placing a braid over a coil, or vice versa. Interweaving the first and second filaments 130, 132 also expedites manufacturing as it can be accomplished in a single process, while overlaying a braid and a coil requires at least two separate manufacturing steps (e.g., braiding a braided tube onto a mandrel, then overlaying a coil, or placing a coil on a mandrel and braiding a braided tube over the coil).

The number of second filaments 132 can be greater than the number of first filaments 130. For example, in some embodiments, the braid 104 can include at least 5, 6, 7, 8, 9 10, 11, 12, 13, 14, 15, 16, 17, 18, 19, or 20 times as many first filaments 130 as second filaments 132. In some (but not all) embodiments of the shaft 104 in which the outer diameter is 8 Fr, the braid 120 can comprise two first filaments 130 and 14 second filaments 132. In some (but not all) embodiments of the shaft 104 in which the outer diameter is 12 Fr, the braid 120 can comprise two first filaments 130 and 30 second filaments 132. Other combinations are possible and within the scope of this disclosure.

In some embodiments, the stiffnesses of the individual first filaments 130 can be different than the stiffnesses of the individual second filaments 132. In such embodiments, the stiffness variation may be achieved by varying the size, shape, thickness, and/or material composition of the filaments 130, 132.

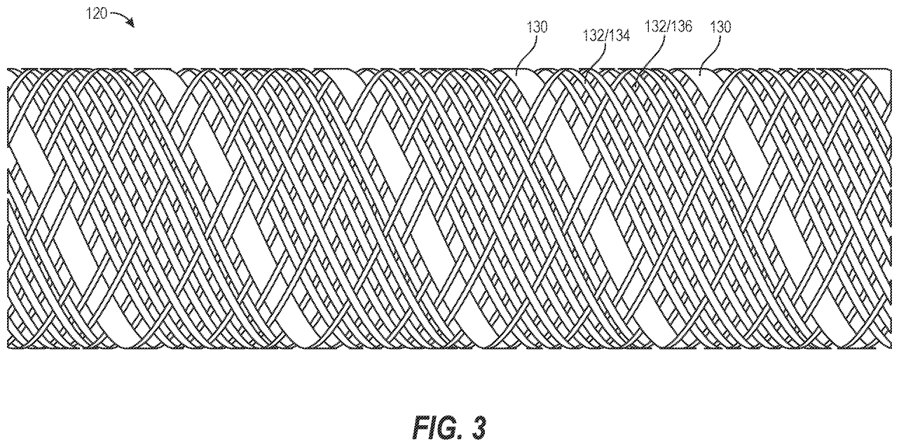

The individual second filaments 132 can have a cross-sectional area that is less than the cross-sectional area of the individual first filaments 130. The larger size of the first filaments 130 beneficially provides greater radial and column strength to the shaft 104. In some embodiments, the first and second filaments 130, 132 have the same cross-sectional area. The first and second filaments 130, 132 can have the same or different cross-sectional shapes (e.g., both circular, both ovular, both rectangular, etc.). For example, as shown in FIGS. 1B and 3, in some embodiments the first and second filaments 130, 132 have a rectangular cross-sectional shape. As shown in FIG. 2, in some variations the first and second filaments 130, 132 have a circular cross-sectional shape. In some embodiments, the first filaments 130 have a circular cross-sectional shape while the second filaments 132 have a rectangular cross-sectional shape, or vice versa. In any case, the filaments 130, 132 can comprise a metal, such as stainless steel, platinum, silver, tantalum, a superelastic and/or shape-memory material (e.g., nitinol, a cobalt chromium alloy, MP35N, 35N LT, etc.), or others. In some embodiments, the filaments 130, 132 can include or be made of non-metallic materials. The first and second filaments 130, 132 can be made of the same or different materials.

In some embodiments, the second filaments 132 comprise a first group 134 wound in the same direction as the first filaments 130 (clockwise or counterclockwise) and that do not cross the first filaments 130, and a second group 136 wound in the opposite direction (clockwise or counterclockwise) as the first group 134 and the first filaments 130 and interwoven with the first group 134 and the first filaments 130. The second group 136 of second filaments 132 can be interwoven with the first group 134 and the first filaments 130 in a 1-over-1 or 2-over-2 pattern, or others. A torque applied to the proximal end portion 104a of the shaft 104 urges the first filaments 130 to radially expand, as the distal ends of the first filaments 130 are fixed at the distal end of the shaft 104. The second filaments 132, however, act as locking members that resist radial expansion of the first filaments 130 and avoid kinking.

The number of second filaments 132 in the first and second groups 134, 136 can be the same or different. In some embodiments, the number of second filaments 132 in the first group 134 is less than the number of second filaments 132 in the second group 136. In some (but not all) embodiments of the shaft 104 in which the outer diameter is 8 Fr, the braid 120 can comprise two first filaments 130, six first group filaments, and eight second group filaments. In some (but not all) embodiments of the shaft 104 in which the outer diameter is 12 Fr, the braid 120 can comprise two first filaments 130, 14 first group 134 filaments, and 16 second group 136 filaments. Other combinations are possible and within the scope of this disclosure.

The catheters 100 of the present technology are configured to be positioned around a turn in a blood vessel (or any tube) having a radius of curvature 24 mm or less and withstand a torque of at least 360 degrees (for example, to direct a bent distal end of the catheter in a certain direction) or at least 12 Nm without the sidewall of the shaft 104 collapsing inwardly (e.g., kinking) at any point along the length of the shaft 104. Said another way, while the distal end of the catheter 100 is distal of a turn in a blood vessel (or any tube) having a radius of curvature 24 mm or less, the lumen 110 of the shaft 104 remains at least 70 percent patent, at least 80 percent patent, at least 90 percent patent, or substantially 100 percent patent while a torque of at least 360 degrees or at least 12 Nm is applied to the sidewall at the proximal end portion 104a of the shaft 104. As such, the catheters 100 of the present technology can be positioned around a turn in a vessel having a radius of curvature of 24 mm or less and receive a second elongate device therethrough (such that a distal end of the second elongate device extends distally of the distal tip of the shaft 104), where the second elongate device has an outer diameter that is at least 70 percent, 80 percent, or 90 percent of the inner diameter of the shaft 104.

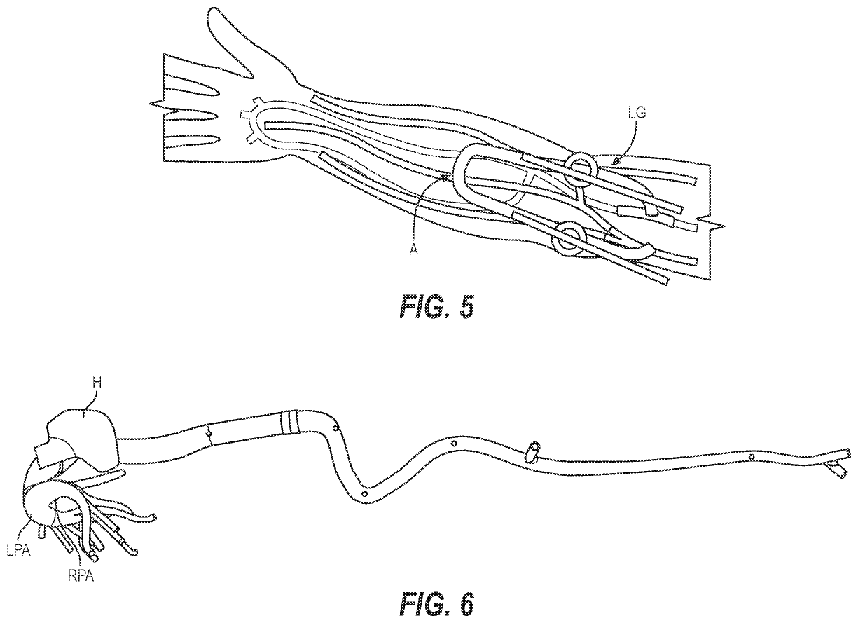

As previously mentioned, the catheters 100 of the present technology can be used in a variety of medical procedures. For example, the catheters 100 of the present technology can be used to remove clot from the peripheral vasculature. An example portion of the lower limb peripheral vasculature is shown in FIG. 4. As shown, the catheter 100 can be tracked contralaterally, e.g., from a femoral access point I, around the femoral access angle θ₁, around the iliac bifurcation C (having a nominal radius of 1.69 inches) and towards Hunter's Canal. The catheter 100 can extend to a position between A and B (to access clot), or distal to B. Other locations within the peripheral vasculature are possible. In such embodiments, the elongate shaft 104 can have an outer diameter of about 0.079 inches (6 French) to about 0.118 inches (9 French), or about 0.105 (8 French).

As another example, the catheter 100 can be configured to track around the apex of an arteriovenous graft (AVG) (or “loop graft”) (e.g., for removal of emboli or other material, or other purposes). An example loop graft LG is shown in FIG. 5. The apex A of the loop graft can have a radius of curvature of about 7 mm to about 100 mm, about 7 mm to about 30 mm, about 10 mm or less, about 20 mm or less, or about 30 mm or less, and a bend angle of at least 30 degrees, or about at least 180 degrees, or about 30 degrees to about 180 degrees. The catheter 100 can access the vessel on one side of the graft and then be tracked around the loop apex A. Other configurations of loop grafts are possible. In such embodiments, the elongate shaft 104 can have an outer diameter of about 0.079 inches (6 French) to about 0.118 inches (9 French), or about 0.105 (8 French).

As yet another example, the catheter 100 can be configured to track and navigate to the pulmonary arteries (for retrieval of pulmonary emboli) typically from femoral access through challenging and tortuous vessel anatomy. An example path is shown in FIG. 6. As shown, the catheter 100 can be advanced through the heart H and around the bifurcation in the main pulmonary artery into the left or right pulmonary artery LPA, RPA. Other locations within the pulmonary arteries are possible. When entering the pulmonary arteries, the catheter 100 can be positioned around a bend having a radius of curvature of about 7 mm to about 100 mm, about 7 mm to about 20 mm, about 7 mm to about 10 mm, or no greater than 30 mm. The shaft 104 can have an outer diameter of about 0.131 inches (10 French) to about 0.315 inches (24 French), about 0.158 inches (12 French) to about 0.315 inches (24 French), about 0.158 inches (12 French) or greater, or about 0.158 inches (12 French).

### CONCLUSION

Although many of the embodiments are described above with respect to systems, devices, and methods for mechanical thrombectomy, the technology is applicable to other applications and/or other approaches, such as removal of unwanted material from other body lumens, or use catheter shafts not intended and/or suitable for aspiration. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-6.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An intravascular catheter, comprising:
an elongate tubular sidewall defining a lumen extending therethrough and having a proximal end, a distal end, and a length between the proximal and distal ends, the sidewall comprising a plurality of filaments, at least some of the filaments being interwoven with other filaments of the plurality of filaments, wherein the catheter is configured to be positioned around a turn in a blood vessel having a radius of curvature no greater than 24 mm, and wherein, while the distal end of the catheter is distal of the turn, the lumen remains at least 70 percent patent while torque applied to the sidewall at the proximal end of the catheter is transmitted to the distal end.

2. The catheter of claim 1, wherein the torque is at least 12 Nm.

3. The catheter of claim 1, wherein the sidewall comprises a longitudinal axis extending along its length, and wherein torque applied at the proximal end of the catheter is configured to rotate the sidewall 360 degrees about the longitudinal axis.

4. The catheter of claim 1, wherein, when the catheter is positioned around the turn and the torque is applied while the lumen remains at least 70 percent patent, an inserted length of the catheter is at least 50 percent.

5. The catheter of claim 1, wherein the sidewall has an outer diameter of at least 8 Fr.

6. The catheter of claim 1, wherein the sidewall has an outer diameter of at least 12 Fr.

7. The catheter of claim 1, wherein the turn in the blood vessel has a bend angle that is greater than or equal to 120 degrees.

8. The catheter of claim 1, wherein the sidewall defines a lumen extending along the longitudinal axis, and wherein the proximal end of the catheter is configured to be fluidly coupled to a negative pressure source to aspirate occlusive material within the blood vessel into and through the lumen.

9. An intravascular catheter, comprising:
a tubular sidewall having a longitudinal axis, the sidewall comprising a first number of first wires and a second number of second wires, the second number greater than the first number, wherein:
the first wires have a first cross-sectional area and are helically wrapped around the longitudinal axis in a first direction without crossing over one another, and
the second wires have a second cross-sectional area less than the first cross-sectional area, wherein the second wires comprise (a) a first group wound in the first direction and that do not cross over the first wires, and (b) a second group wound in a second direction opposite the first direction, and wherein the wires in the second group are interwoven with the first wires and the first group of the second wires.

10. The catheter of claim 9, wherein the second number is at least five times greater than the first number.

11. The catheter of claim 9, wherein a cross-sectional shape of the first wires is different than a cross-sectional shape of the second wires.

12. The catheter of claim 9, wherein the first wires have a rectangular cross-sectional shape and the second wires have a circular cross-sectional shape.

13. The catheter of claim 9, wherein the sidewall defines a lumen, and wherein a proximal end of the catheter is configured to be fluidly coupled to a negative pressure source to aspirate occlusive material within the blood vessel into and through the lumen.

14. An intravascular catheter, comprising:
a proximal end, a distal end, and a longitudinal axis extending therebetween;
a tubular sidewall defining a lumen, the sidewall comprising a plurality of wires embedded in a material, the plurality of wires comprising a first number of first wires and a second number of second wires, the second number greater than the first number, wherein:
the first wires are helically wrapped around the longitudinal axis in a first direction without crossing over one another, the first wires being configured to resist radial collapse of the sidewall, and
the second wires comprise (a) a first group wound in the first direction and that do not cross over the first wires, and (b) a second group wound in a second direction opposite the first direction, wherein the second wires are configured to engage the first wires to resist radial expansion of the first wires, thereby providing improved torqability of the sidewall,
wherein the proximal end of the catheter is configured to be fluidly coupled to a negative pressure source for application of suction through the lumen.

15. The catheter of claim 14, wherein the material is a first material and the plurality of wires and first material together comprise a first layer, and wherein the sidewall further comprises a second layer radially inward of the first layer.

16. The catheter of claim 14, wherein the material is a first material and the plurality of wires and first material together comprise a first layer, and wherein the sidewall further comprises a second layer radially outward of the first layer.

17. The catheter of claim 14, wherein the material is a first material and the plurality of wires and first material together comprise a first layer, and wherein the sidewall further comprises a second layer radially inward of the first layer and a third layer radially outward of the first layer.

18. The catheter of claim 14, wherein a proximal end of the catheter is configured to be fluidly coupled to a negative pressure source to aspirate occlusive material within the blood vessel into and through the lumen.

19. The catheter of claim 14, wherein the tubular sidewall has an outer diameter of at least 8 Fr.

20. The catheter of claim 14, wherein the tubular sidewall has an outer diameter of at least 12 Fr.
